(19) United States
(12) Patent Application Publication
(10) Pub. No.: US 2008/0228276 A1
(43) Pub. Date: Sep. 18, 2008

(54) Intervertebral Prosthesis, Instruments, and Methods of Implanting

(75) Inventors: Hallett Mathews, Williamsburg, VA (US); Mingyan Liu, Bourg la Reine (FR); Jeffrey Zhang, Collierville, TN (US); Chris Hughes, Lakeland, TN (US); Lukas G. Eisermann, San Diego, CA (US); Jean-Charles LeHuec, Pessac (FR)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/686,206

(22) Filed: Mar. 14, 2007

(51) Int. Cl. A61F 2/44; A61B 17/58

(52) U.S. Cl. 623/17.16; 606/99

(57) ABSTRACT

A method of implanting an intervertebral prosthesis in an intervertebral space between a first and a second vertebra of a patient's spinal column may include making a first aperture at a first location to permit access to the intervertebral disc space. The first aperture may be sized to allow passage of at least a portion of the intervertebral prosthesis. A second aperture may be made at a second location to permit access to the intervertebral disc space. The second aperture may be sized to allow passage of a surgical instrument. An intervertebral prosthesis for implantation into an intervertebral space between a first and a second vertebra of a patient's spinal column is also disclosed, along with a surgical instrument for implanting the an intervertebral prosthesis.

### FIELD OF THE INVENTION

This disclosure is directed toward generally to a prosthesis, instruments, and methods of implanting the prosthesis, and more particularly, to an intervertebral disc, instruments and methods of implanting the intervertebral disc in an intervertebral space.

### BACKGROUND

Disc arthroplasty is one way of treating injured, degraded, or diseased spinal joints. Some disc arthroplasty treatments include replacing injured discs of the joint with a motion-preserving spinal disc that allows some articulation or movement of the spinal joint. While the inserted disc may provide joint articulation to a patient, inserting the spinal disc can be an invasive and intensive procedure. For example, conventional spinal discs are installed anteriorly. Because anterior procedures often require displacement of organs, such as the aorta and vena cava, they must be performed with great care. Further, because scar tissue may grow about the surgical site, any required second treatment can be more difficult, and may introduce additional distress to the patient.

What is needed is an intervertebral prosthesis, instruments, and a surgical method for inserting the intervertebral prosthesis into an intervertebral space using a generally posterior transforaminal approach, a lateral approach, and/or a posterior lateral approach. The method of implanting an artificial disc disclosed herein overcomes one or more problems in the prior art.

### SUMMARY OF THE INVENTION

In one exemplary aspect, the present disclosure is directed toward a method of implanting an intervertebral prosthesis in an intervertebral space between a first and a second vertebra of a patient's spinal column. The method may include making a first aperture at a first location to permit access to the intervertebral disc space. The first aperture may be sized to allow passage of at least a portion of the intervertebral prosthesis. A second aperture may be made at a second location to permit access to the intervertebral disc space. The second aperture may be sized to allow passage of a surgical instrument.

In one aspect, the second aperture is smaller than the first aperture.

In another aspect, the intervertebral prosthesis may be introduced into the intervertebral space through the first aperture.

In yet another aspect, the intervertebral space may be accessed with the surgical instrument through the second aperture.

In another aspect, the surgical instrument may connect to the intervertebral prosthesis through the second aperture.

In another aspect, the present disclosure is directed toward a method of implanting an intervertebral prosthesis in an intervertebral space between a first and a second vertebra of a patient's spinal column by making a first aperture at a first location laterally offset on a first side of a midline of the patient's spinal column. The first aperture may be configured to permit access to the intervertebral disc space and may be sized to allow passage of at least a portion of the intervertebral prosthesis. A second aperture may be made at a second location laterally offset on a second side of the midline of the patient's spinal column. The second side may be opposite the first side. The second aperture may be configured to permit access to the intervertebral disc space and may be sized to allow passage of an instrument. In addition, the second aperture may be smaller than the first aperture. A surgical instrument may be connected to the intervertebral prosthesis through the second aperture, and the intervertebral prosthesis may be inserted into the intervertebral space through the first aperture.

In yet another aspect, the present disclosure is directed to an intervertebral prosthesis for implantation into an intervertebral space between a first and a second vertebra of a patient's spinal column. The prosthesis may include a first end and a second end. A first bearing surface may extend between the first and second ends and may be configured to bear weight transferred from the first vertebra of the spinal column. A second bearing surface may be disposed opposite the first bearing surface and may be configured to transfer weight to the second vertebra of the spinal column. A first connecting element may be configured to cooperate with a surgical instrument operable from the first end of the intervertebral prosthesis. A second connecting element may be configured to cooperate with a surgical instrument operable from the second end of the intervertebral prosthesis.

In yet another aspect, a surgical instrument for implanting an intervertebral prosthesis in an intervertebral space between a first and a second vertebra of a patient's spinal column may be disclosed. The surgical instrument may include a distal end having a connector configured to engage the intervertebral prosthesis and pull the intervertebral prosthesis at least partially within the intervertebral disc space. A proximal end may be configured to be manipulated by a physician. The distal end may be responsive to the manipulation to pull the intervertebral prosthesis. A body may extend between the distal and proximal ends.

### BRIEF DESCRIPTION OF THE DRAWINGS

FIG. 1 is a pictorial representation of a side view of the lumbar spinal and the sacrum regions of a healthy, human spinal column.

FIG. 2 is a pictorial representation of a portion of a right side of a lumbar vertebrae with a healthy disc disposed between two vertebrae.

FIG. 3 is a pictorial representation of an isometric view of an articulating intervertebral prosthesis for implantation according to one embodiment of the present disclosure.

FIG. 4 is a pictorial representation of a lateral, partial sectional view of the intervertebral prosthesis of FIG. 3 disposed between a pair of adjacent vertebrae.

FIG. 5 is a pictorial representation of a top view of a lower vertebra.

FIG. 6 is a pictorial representation of a top view of a lower vertebra undergoing a disc removal step of a disc replacement procedure.

FIG. 7 is a pictorial representation of a top view of a lower vertebra undergoing a disc insertion step of a disc replacement procedure.

FIG. 8 is a pictorial representation of a top view of a lower vertebra undergoing a further disc insertion step of a disc replacement procedure.

FIG. 9 is a pictorial representation of a top view of a lower vertebra with an inserted disc disposed in a working location.

### DETAILED DESCRIPTION

The present invention relates generally to a method and system of vertebral reconstruction, and more particularly, to a method and system for inserting an artificial intervertebral disc or prosthesis in an intervertebral space. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

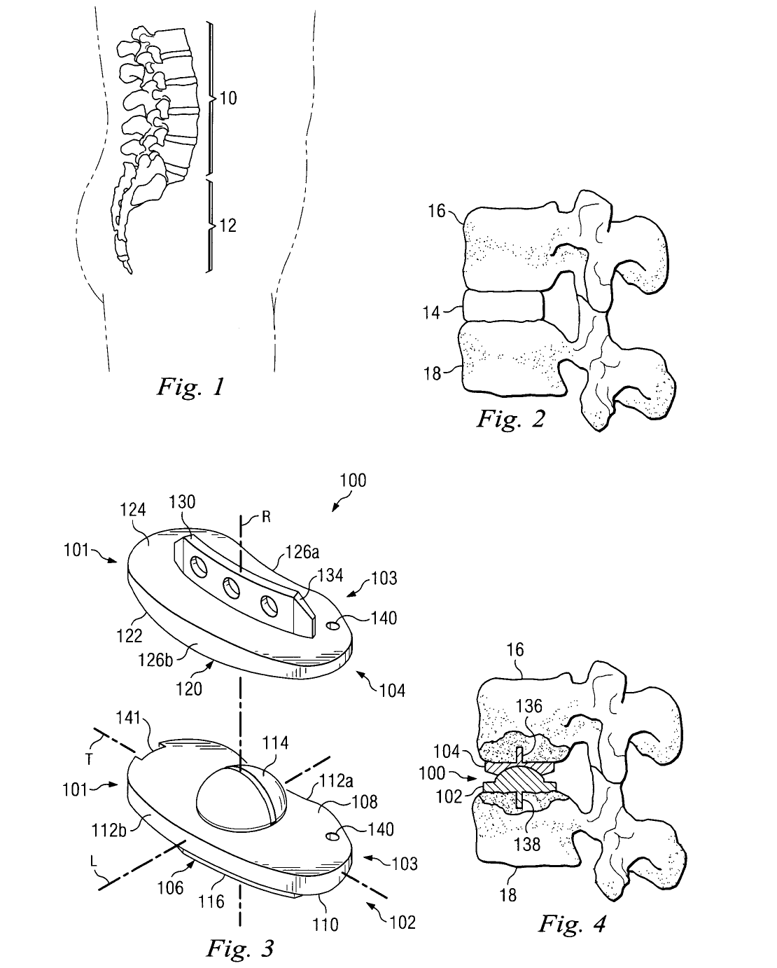

FIG. 1 illustrates schematically the lumbar spinal 10 and the sacrum regions 12 of a healthy, human spinal column. The spinal regions are made up of vertebrae separated by intervertebral discs. For the sake of example, two of the vertebrae will be discussed with reference to FIG. 2. FIG. 2 illustrates a portion of the right side of a lumbar spinal region with a healthy intervertebral disc 14 disposed between two adjacent vertebrae 16, 18. In any given joint, the top vertebra may be referred to as the superior vertebra and the bottom one as the inferior vertebra.

Conventional spinal prosthetic devices are implanted using an anterior procedure, requiring a physician to access the spinal column using distressing and sometimes traumatic procedures. Once accessed, some types of disc arthroplasty require that some or all of the natural disc that would have been positioned between the two vertebrae 16, 18 be removed via a discectomy or a similar surgical procedure. Removal of the diseased or degenerated disc results in the formation of an intervertebral space between the upper and lower vertebrae 16, 18. A prosthetic vertebral disc may be installed in the intervertebral space, replacing the natural disc. Once a prosthetic vertebral disc is installed using an anterior procedure, scar tissue may quickly build on sensitive and important organs. If a second procedure is required, a physician is required to remove the scar tissue to access the previously placed prosthetic. This procedure can be sensitive and can cause distress to the patient. The methods and devices for implanting an artificial intervertebral disc disclosed herein may be advantageous over prior methods and devices because it may be installed using a posterior transforaminal approach, a lateral approach, or a posterior lateral approach, rather than an anterior one. Accordingly, a physician need not access and disturb the critical organs that reside anteriorly of the spinal column. Further, if a second procedure becomes necessary, the physician has easy access to the previously placed prosthesis without removing scar tissue off of sensitive organs. Accordingly, the procedure may be simplified and may cause less distress to the patient.

FIGS. 3 and 4 illustrate an exemplary artificial intervertebral disc 100 suitable for use with the implantation method disclosed herein. It should be noted that the artificial intervertebral disc 100 is only one example, and that any other suitable disc may be used. Turning to FIGS. 3 and 4, the artificial intervertebral disc 100 extends generally along a longitudinal axis L and includes a first articular component 102 and a second articular component 104, that extend between a first end 101 and a second end 103. The articular components 102, 104 cooperate to form the artificial intervertebral disc 100 which is sized and configured for disposition within an intervertebral space between the adjacent vertebrae 16, 18 of FIG. 2. FIG. 4 is an exemplary partial cross-sectional view showing the artificial intervertebral disc 100 filling the intervertebral space between the vertebrae 16, 18.

The artificial intervertebral disc 100 may provide relative pivotal and rotational movement between the adjacent vertebrae 16, 18 to maintain or restore relative motion. Preferably, the maintained or restored motion is substantially similar to the normal bio-mechanical motion provided by the natural intervertebral disc 14 of FIG. 2. More specifically, the articular components 102, 104 are permitted to pivot relative to one another about a number of axes, including lateral or side-to-side pivotal movement about longitudinal axis L and anterior-posterior pivotal movement about a transverse axis T. It should be understood that in some embodiments, the articular components 102, 104 may pivot relative to one another about any axes that lies in a plane that intersects longitudinal axis L and transverse axis T. Additionally, the articular components 102, 104 are preferably permitted to rotate relative to one another about a rotational axis R. Although the artificial intervertebral disc 100 is illustrated and described as providing a specific combination of articulating motion, it should be understood that other combinations of movement, including movement supplied by non-articulating prosthetic discs, are also possible and are contemplated as falling within the scope of the present disclosure. It should also be understood that other types of articulating movement are also contemplated, such as, for example, relative translational or linear motion.

The articular components 102, 104 of artificial intervertebral disc 100 may be formed of any suitable biocompatible material including, for example, metals such as cobalt-chromium alloys, titanium alloys, nickel titanium alloys, and/or stainless steel alloys. In one embodiment, the articular components 102, 104 are formed of a cobalt-chrome-molybdenum metallic alloy (ASTM F-799 or F-75). Ceramic materials such as aluminum oxide or alumina, zirconium oxide or zirconium, compact of particulate diamond, and/or pyrolytic carbon may also be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE, among others. The various components comprising the arthroplasty halves 102, 104 may be formed of different materials thus permitting metal on metal, metal on ceramic, metal on polymer, ceramic on ceramic, ceramic on polymer, or polymer on polymer constructions.

The surfaces of the articular components 102, 104 that are positioned in direct contact with vertebral bone may include features or coatings which enhance the fixation of the implanted prosthesis. In one example, the surfaces of the articular components 102, 104 may be coated with a bone-growth promoting substance, such as, for example, a hydroxyapatite coating formed of calcium phosphate, tricalcium phosphate (TCP), and/or calcium carbonate. Alternatively, osteoinductive coatings, such as proteins from transforming growth factor (TGF) beta superfamily, or bone-morphogenic proteins, such as BMP2 or BMP7, may be used. In some embodiments, the surfaces of the articular components 102, 104 that are positioned in direct contact with vertebral bone are roughened prior to being coated with the bone-growth promoting substance to further enhance bone growth. Such surface roughening may be accomplished by way of, for example, acid etching, knurling, application of a bead coating, chemical etching, bead-blasting, sanding, grinding, serrating, and/or diamond-cutting or other methods of roughening that would occur to one of ordinary skill in the art. Other suitable features may include spikes, ridges, and/or other surface textures.

The articular component 102 includes a support plate 106 having an inner surface 108 and an opposite bearing surface 110. The support plate 106 may be sized and shaped to substantially correspond to the size and shape of the vertebral endplate of an adjacent vertebra. In one embodiment, the support plate 106 is shaped to facilitate a posterior transforaminal approach, a lateral approach, or a posterior lateral approach. As such, the support plate 106 includes curved side portions 112a, 112b, which are defined as the generally elongated portions of the support plate 106 extending between the inner surface 108 and the bearing surface 110. Although not shown, the support plate 106 can include one or more notches or other types of indicia for receiving and engaging with a corresponding portion of a surgical instrument (also not shown) to aid in the manipulation and insertion of the artificial intervertebral disc 100 within an intervertebral space between adjacent vertebrae. The surgical instrument (not shown) is preferably configured to hold the articular components 102, 104 at a predetermined orientation and spatial relationship relative to one another during manipulation and insertion of the artificial intervertebral disc 100, and to release the articular components 102, 104 once properly positioned between the adjacent vertebrae.

In one embodiment, the inner surface 108 includes a projection 114 having a convex shape, which may be configured as a spherical-shaped ball (half of which is shown). It should be understood that other configurations of the projection 114 are also contemplated, such as, for example, cylindrical, elliptical or other arcuate configurations or possibly non-arcuate configurations. It should also be understood that the remaining portion of inner surface 108 may take on planar or non-planar configurations, such as, for example, an angular or conical configuration extending about the projection 114.

The articular component 104 shown in FIG. 3 includes a support plate 120 having an inner surface 122 and an opposite bearing surface 124. The support plate 120 may be sized and shaped to substantially correspond to the size and shape of the vertebral endplate of an adjacent vertebra. The support plate 106 may include curved side portions 126a, 126b, which correspond to the side portions 112a, 112b of the articular component 102. Also, like the support plate 106, the support plate 120 also may include one or more notches or indicia for receiving and engaging a surgical instrument.

In one embodiment, the inner surface 122 includes a recess (not shown) having a profile that matches the profile of the projection 114. The recess may be configured to receive the projection 114, and form a ball and socket joint. It should be understood that the joint may be formed of other configurations, such as, for example, cylindrical, elliptical or other arcuate configurations or possibly non-arcuate configurations.

In the embodiment shown in FIG. 3, the articular components 102, 104 of the artificial intervertebral disc 100 each include a first connecting element 140 for connection to a first surgical tool (described further below) and a second connecting element 141 for connection to a second surgical tool (not shown). In the embodiment shown, the connecting element 140 is a hole formed in each of the top and bottom support plates 106, 120. The connecting element 140 may be disposed adjacent the second end 103, which may be configured as a leading edge of the disc 100 during implantation. Accordingly, the connecting element 140 may be used during implantation to connect to the first surgical tool, which may then be used to pull or otherwise adjust the artificial intervertebral disc 100 into position within the intervertebral space. Although shown as a hole in FIG. 3, the connecting element 140 could be any feature configured to connect to the surgical tool. For example, the connection element could be a hook, a tab, a protrusion, an indentation, among other things. In addition, it need not be formed on the top and bottom support plates 106, 120, but may be formed anywhere on the intervertebral disc 100, so long as the connection element can be accessed during implantation to manipulate the disc. In some embodiments, the connecting element 140 is formed along the outer peripheral edge of the disc.

At the same time, the second connecting element 141 may be configured to connect to a surgical instrument configured to operate from the first end 101, or a trailing end, of the disc to push or manipulate the disc 100 into the intervertebral disc space. Such tools that aid in implantation by pushing from a trailing end are known in the art, and therefore, are not explained or shown in detail.

Flange members or keels 116, 130 extend from the respective bearing surfaces 110, 124 and may be configured for disposition within preformed openings in the respective adjacent vertebral endplates. In one embodiment, the keels 116, 130 extend perpendicularly from the bearing surface 110, 124 and are approximately centrally located along the bearing surface 110, 124. In some embodiments however, the position of the keels 116, 130 may be offset to help circumvent veins, arteries, bony portions, or other obstacles that may be in place during the insertion of the intervertebral disc 100.

In one embodiment, the keels 116, 130 transversely extend along a substantial portion of the support plate 106, 120. The keels 116, 130 may have a curved profile that in some examples is substantially similar to and congruous with the degree of curvature of the side portions 112a, 112b, 126a, 126b. Such an embodiment would accommodate insertion of the artificial intervertebral disc 100 using a posterior transforaminal approach, a lateral approach, or a posterior lateral approach, as opposed to the anterior approach discussed above. In a further embodiment, the keels 116, 130 may be angled, tapered, or configured in some other shape to facilitate the functional demands of the keel. In still another embodiment, the keels 116, 130 may be configured as a winged keel, including a transverse portion (not shown) extending across the main body portion of the keels.

In the embodiment shown in FIG. 3, the keels 116, 130 include three openings 132 extending therethrough to facilitate bone through-growth to enhance fixation to the adjacent vertebra. However, it should be understood that any number of openings 132 may be defined through the keels 116, 130, including a single opening or two or more openings. In some embodiments, there are no openings at all. It should also be understood that the openings 132 need not necessarily extend entirely through the keels 116, 130 but may alternatively extend partially therethrough. As discussed above, the surfaces of the articular component 102, 104 that are in direct contact with vertebral bone are preferably coated with a bone-growth promoting substance, such as, for example, hydroxyapatite. Specifically, the bearing surfaces 110, 124 and the surfaces of the keels 116, 130 may be coated with the substance to promote bony engagement with the adjacent vertebrae. As also discussed above, the bearing surface 110, 124 and the surfaces of the keels 116, 130 may be roughened prior to application of the hydroxyapatite coating.

In some embodiments, one or both of the keels 116, 130 may include a sharp forward edge, illustrated by edge 134 on the keel 130. By having such an edge, insertion of the keel into the associated vertebral body is facilitated. Also, the edges 134 can be of sufficient sharpness that the vertebral bodies do not require a slot for receiving the keels 116, 130, discussed in greater detail below. In some examples, the artificial intervertebral disc 100 includes only one of the keels 116, 130. Accordingly, the bearing surface 110, 124 not including a keel may be smooth or flat, or may include other features. In other embodiments, the artificial intervertebral disc 100 does not include any keels. When included, the keels 116, 130 need not have similar features, but may differ in relative location and structure, as would be apparent to one skilled in the art.

Referring to FIG. 4, to accommodate insertion of the artificial intervertebral disc 100 within the intervertebral space, the upper and lower vertebrae 16, 18 can be prepared to accept the artificial intervertebral disc 100 therebetween. For example, multiple slots 136, 138 may be formed along the vertebral endplates of the upper vertebrae 16 and the lower vertebrae 18. The slots 136, 138 may be created by the keels 116, 130 themselves, or can be prepared beforehand.

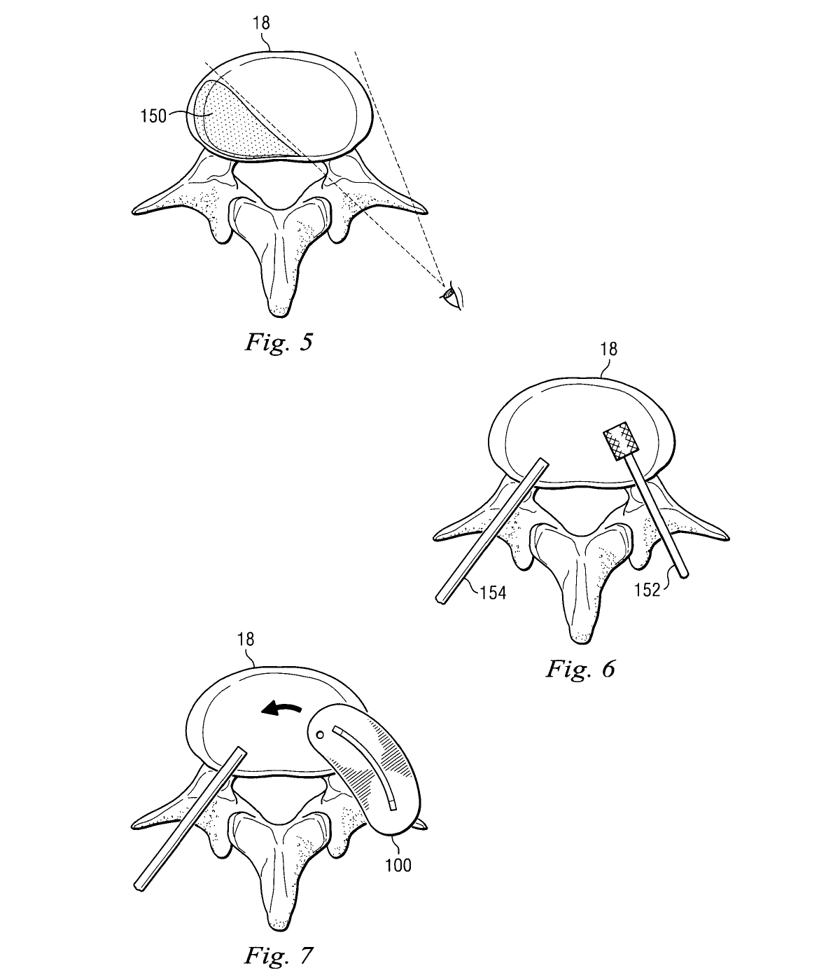

An exemplary procedure for implanting the artificial intervertebral disc 100 between the vertebrae 16, 18 will now be described below with reference to FIGS. 5-9. FIGS. 5-9 each show a top view of the lower vertebra 18. Generally, as discussed above, the artificial intervertebral disc 100 may be implanted into a body using a posterior transforaminal approach, a lateral approach, and a posterior lateral approach.

According to one example of the implantation procedure, a first incision or aperture may be made in the patient's back to provide access to the relevant vertebrae and spinal disc. The procedure will be described as though the first surgical aperture is made at the right side of a patient's midline, which may be defined by the patient's vertebral column. The first incision or approach could be part of an open procedure or a part of a procedure using a tube or retractor based technique. While the tube could be any suitable size, one exemplary tube is sized to have a diameter within the range of about 22-25 mm. The first incision, whether incorporating a tube or not, should be large enough to enable passage of the artificial intervertebral disc 100 into a space between the vertebrae. From the first incision, a physician may have a line-of-sight view as shown in FIG. 5. Because of the location of spinal vessels and neurological structures, only a portion of the endplates of the vertebrae and the associated intervertebral space can be seen from the first incision. This also creates a blind zone 150, not directly viewable from the first incision.

In order to provide orientation and aid during subsequent steps of the procedure, a second incision or aperture, relatively small when compared to the first incision, may be made on the side of the midline opposite the first incision, or in this case, on the left side of the patient's midline. The second incision may be only a small diameter stab wound. In one example, the second incision has a diameter of about 7 mm. Through the second incision, a small tube may be inserted to prop open the space. Alternatively, an endoscope may be placed directly in the absence of a tube. When a tube is placed, the endoscope may be inserted through the tube. The second incision, therefore, provides access to the area of the blind zone, as shown in FIG. 6.

The present procedure differs from traditional transforaminal approaches because traditional transforaminal approaches have been purely unilateral approaches. When a traditional transforaminal approach is used, the incision is often larger than would occur using the present methods. Further, the present procedure differs from the traditional posterior approach because traditional posterior approaches have been either open or bilateral approaches with relatively equal sized incisions on each side. In contrast to the traditional transforaminal and posterior approaches, the present procedure opens one side of the spine a sufficient amount to provide traditional access, but opens the second side only in a micro-invasive manner. In some examples, the second incision is a stab wound, rather than a sized incision. Accordingly, the procedure is less invasive than prior procedures, reducing trauma to the patient and potentially less distressful to the spinal segment. Further, the present procedure provides better access to the intra-discal tissue than the purely unilateral approach of the traditional transforaminal procedure. In addition, the present procedure provides a much smaller surgical access port on one side than the traditional posterior procedure.

Once the incisions are made, and the muscle and ligaments are moved aside, some or all of the affected natural disc and surrounding tissue may be removed via the first incision. This may done using methods known in the art, using conventional tools sized to fit through the first incision. FIG. 6 shows one example of the disc removal process with a surgical tool 152 accessing the intervertebral space from the right side through the first incision and a tube 154 accessing the intervertebral space from the left side through the second incision.

In a traditional transforaminal approach, the left posterior corner of the intra-discal space is difficult to reliably reach, as it is within the blind zone 150 of FIG. 5. In order to assist with clearing the blind zone of the discal tissue, the blind zone may be visualized with the endoscope. If visualization alone is not enough, the endoscope may be complemented or replaced by a burr or other tissue removal tool.

Because of the difficulty in viewing the blind zone when performing procedures using traditional transforaminal approaches, sometimes the vertebral space is not sufficiently cleared of discal tissue. Because of this, when the artificial intervertebral disc is placed, it may not seat as well as it otherwise would. The present approach, where the progress of the discal tissue removal process may be viewed and monitored, provides a better cleaning and prepping of the vertebrae, resulting in a better seat of the artificial intervertebral disc 100.

Once the discal tissue is removed, or alternatively, while removing the discal tissue, the superior endplate surface of the vertebra 18 may be milled, rasped, or otherwise resected to match the profile of the bearing surface 110 of the artificial intervertebral disc 100. A good match may normalize stress distributions on the endplate surface of the vertebra 18, and/or provide initial fixation prior to bone ingrowth. The preparation of the endplate of vertebra 18 may result in a flattened surface or in surface contours such as pockets, grooves, or other contours that may match corresponding features on the disc 100. The endplate of the vertebra 16 may be similarly prepared to fit the bearing surface 124. The natural facet joints of vertebrae 16, 18 may be trimmed if necessary to provide an access path for the intervertebral disc 100.

If the disc includes keels, as does the artificial intervertebral disc of FIG. 3, then in some examples, the endplates of the vertebrae 16, 18 may be milled, chiseled, or otherwise prepared to create channels that receives the keels 116, 130. The channels may be curved in accordance with the curved keels 116, 130 to facilitate the movement of the artificial intervertebral disc 100 during insertion. In one example, as an alternative to chiseling, a milling guide (not shown) may be used in conjunction with a milling tool to cut the channels in the upper and lower vertebral bone 16, 18. The milling tool may find especial utility when creating curved channels. The keels 116, 130 may help to connect to the bone and limit movement of the articular components 102, 104 of the artificial intervertebral disc 100.

Once the vertebrae 16, 18 are prepared, the artificial intervertebral disc 100 may be inserted through the first incision into the intervertebral space, as shown in FIG. 7. As discussed above, the support plate 106 of the artificial intervertebral disc 100 may be configured to removably receive or engage a corresponding portion of a surgical instrument (not shown) that aids in the manipulation and insertion of the artificial intervertebral disc 100. The articular components 102, 104 may be joined together for simultaneous insertion or may be inserted separately, one at a time.

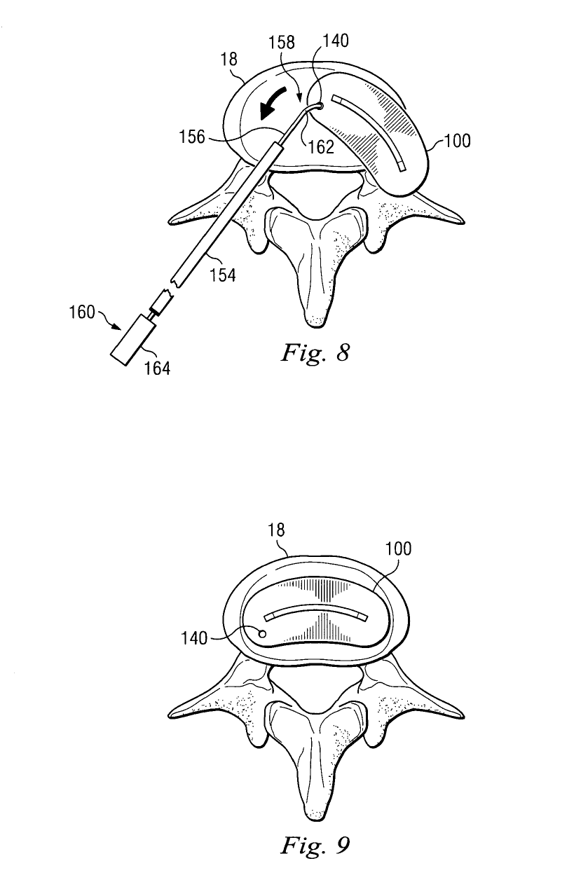

During insertion, it is possible for the artificial disc to become stuck part way into the intervertebral space, particularly in the examples where keels are employed. If this occurs, and as shown in FIG. 8, a surgical tool 156 may be inserted through the second small incision to access the first connecting element 140 on the second end 103 or on the leading edge of the artificial intervertebral disc 100.

The surgical tool 156 may include a distal end 158 and a proximal end 160. The distal end 156 may be configured with a connector 162 configured to cooperate with the first connecting element 140 on the intervertebral disc 100. The connector 162 may be configured to engage the intervertebral disc 100 in a manner to allow the disc to be pulled by the surgical tool 156 and may be, for example, a hook or finger. The proximal end 160 of the surgical tool 156 may include a handle 164 that may be held and/or manipulated by a physician performing the disc replacement procedure. As shown in FIG. 8, the surgical tool may connect to the connecting element 140 at the second end 103 of the intervertebral disc 100, and may be used to pull the disc into the intervertebral space. At the same time, the second tool (not shown) may be used to engage the second connecting element 141 to push the disc 100 from the first end 101, or from the trailing end.

The surgical tool 156 may be a strong cable, thread, or similar leader that is configured to pass through the tube 154, into the intervertebral space, and removably connect with the first connecting element 140. The surgical tool 156 may be any component configured to attach to and pull an intervertebral disc from one side of an intervertebral disc space toward another and, in some examples, includes a hook, pincers, a bend or other configuration capable of operating to attach to the connecting element 140. In some examples, the surgical tool 156 is a rigid, curved cable, while in other examples, the tool is semi-rigid. In others, the surgical tool is flexible.

Once the surgical tool 156 is connected to the intervertebral disc 100, the tool may used to pull the disc into place. In some examples, the surgical tool 156 is used to pull the disc through the second incision at the same time that the disc is being pushed into the intervertebral space through the first incision by the connecting element 141. Pulling and pushing on the intervertebral disc 100 may greatly simplify the insertion process. Further, it may simplify moving and positioning the artificial intervertebral disc 100 after it is fully within the intervertebral space. Once the artificial intervertebral disc 100 is properly positioned in the intervertebral space, any bone fixation elements, such as screws, may be introduced, and the surgical tool 156 may be removed and the tube withdrawn, as shown in FIG. 9.

The procedure disclosed herein allows for a better fit than traditional transforaminal procedures by providing access to the blind region, previously unmonitored during traditional transforaminal procedures. This enables the vertebral space to be better prepared to receive the intervertebral disc 100, making a better fit, and potentially reducing subsequent pain and trauma. Furthermore, it enables the disc to be guided into place from a leading edge, rather than just from the rear, as in traditional approaches. Accordingly, the disc may be implanted with less force and with less difficulty than when using traditional transforaminal approaches.

In addition, the procedure disclosed herein is less invasive than traditional posterior procedures because the second incision for the tube is relatively smaller than the first incision for the disc. Accordingly, the procedure may reduce the amount of trauma and distress occurring with traditional posterior procedures.

Because of its convenient nature, suppliers of the intervertebral disc 100 may sell the device in a kit. A kit may include, for example, the intervertebral disc 100 with the surgical tool 156 that is engagable with the first connection element 140 to pull the disc during implantation. The first connection 140 may be located on the leading end. In addition, the kit may include a second surgical tool engagable with the second connection element 141 to push the intervertebral prosthesis in a manner known in the art. Other combinations could also be used.

Although the illustration of FIG. 2 generally depicts the vertebral joint 12 as a lumbar vertebral joint, it is understood that the devices, systems, and methods of this disclosure may also be applied to all regions of the vertebral column, including the cervical and thoracic regions. In addition, although the artificial intervertebral disc 100 described herein is an articulating disc, the disc could be any prosthetic disc, including a non-articulating disc, such as a disc having an elastomeric core.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “cephalad,” “caudal,” “upper,” and “lower,” are for illustrative purposes only and can be varied within the scope of the disclosure. In the claims, means-plus-function clauses are intended to cover the elements described herein as performing the recited function and not only structural equivalents, but also equivalent elements.

We claim:

1. A method of implanting an intervertebral prosthesis in an intervertebral space between a first and a second vertebra of a patient's spinal column, comprising:
making a first aperture at a first location to permit access to the intervertebral disc space, the first aperture being sized to allow passage of at least a portion of the intervertebral prosthesis;
making a second aperture at a second location to permit access to the intervertebral disc space, the second aperture being sized to allow passage of a surgical instrument, the second aperture being smaller than the first aperture;
introducing the intervertebral prosthesis into the intervertebral space through the first aperture; and
accessing the intervertebral prosthesis with the surgical instrument through the second aperture.

2. The method of claim 1, wherein the first and second apertures are on opposite sides of a midline defined by the patient's spinal column.

3. The method of claim 1, including:
connecting the surgical instrument to the intervertebral prosthesis; and
manipulating the intervertebral prosthesis with the instrument.

4. The method of claim 3, wherein manipulating the intervertebral prosthesis includes pulling the prosthesis with the surgical instrument.

5. The method of claim 1, wherein the surgical instrument is flexible.

6. The method of claim 1, wherein the surgical instrument includes a hook formed at one end.

7. The method of claim 1, wherein the surgical instrument is an endoscope and wherein accessing the intervertebral space includes inserting the endoscope through the second aperture.

8. The method of claim 7, including:
removing tissue from the intervertebral space through the first aperture; and
monitoring the tissue removal with the endoscope in the second aperture.

9. The method of claim 1, including inserting a tube into the second aperture, and wherein accessing the intervertebral prosthesis includes introducing the surgical instrument through the tube.

10. The method of claim 9, including inserting an endoscope through the tube.

11. The method of claim 9, including removing tissue from the intervertebral space through the tube.

12. The method of claim 1, wherein the second aperture is too small to allow passage of the intervertebral prosthesis.

13. The method of claim 1, wherein the first aperture is made at one of the left and right sides of the first and second vertebrae and the second aperture is made at the other of the left and right sides of the first and second vertebrae.

14. A method of implanting an intervertebral prosthesis in an intervertebral space between a first and a second vertebra of a patient's spinal column, comprising:
making a first aperture at a first location to permit access to the intervertebral disc space, the first aperture being sized to allow passage of at least a portion of the intervertebral prosthesis;
making a second aperture at a second location to permit access to the intervertebral disc space, the second aperture being sized to allow passage of a surgical instrument;
connecting the surgical instrument to the intervertebral prosthesis through the second aperture; and
introducing the intervertebral prosthesis to the patient through the first aperture.

15. The method of claim 14, wherein the first and second apertures are on opposite sides of a midline of the patient's spinal column.

15. The method of claim 14, wherein the second aperture is smaller than the first aperture.

16. The method of claim 14, wherein introducing the intervertebral prosthesis includes pulling the prosthesis with the surgical instrument.

17. The method of claim 14, wherein the surgical instrument is flexible.

18. The method of claim 14, wherein the surgical instrument includes a hook formed at one end.

19. The method of claim 14, including accessing the intervertebral disc space with an endoscope through the second aperture.

20. The method of claim 19, including:
removing tissue from the intervertebral space through the first aperture; and
monitoring the tissue removal with the endoscope in the second aperture.

21. The method of claim 14, including accessing the intervertebral disc space with a tube inserted into the second aperture.

22. The method of claim 21, including removing tissue from the intervertebral space through the tube.

23. The method of claim 14, wherein the second aperture is too small to allow passage of the intervertebral prosthesis.

24. A method of implanting an intervertebral prosthesis in an intervertebral space between a first and a second vertebra of a patient's spinal column, comprising:
making a first aperture at a first location laterally offset on a first side of a midline of the patient's spinal column, the first aperture being configured to permit access to the intervertebral disc space and being sized to allow passage of at least a portion of the intervertebral prosthesis;
making a second aperture at a second location laterally offset on a second side of the midline of the patient's spinal column, the second side being opposite the first side, the second aperture being configured to permit access to the intervertebral disc space and being sized to allow passage of an instrument, the second aperture being smaller than the first aperture;
connecting a surgical instrument to the intervertebral prosthesis through the second aperture; and
inserting the intervertebral prosthesis into the intervertebral space through the first aperture.

25. The method of claim 24, comprising manipulating the intervertebral prosthesis by pulling the prosthesis with the surgical instrument.

26. The method of claim 24, including inserting an endoscope through the second aperture.

27. The method of claim 26, including:
removing tissue from the intervertebral space through the first aperture; and
monitoring the tissue removal with the endoscope in the second aperture.

28. The method of claim 24, including inserting a tube into the second aperture.

29. The method of claim 28, including inserting an endoscope through the tube.

30. The method of claim 24, wherein the second aperture is too small to allow passage of the intervertebral prosthesis.

31. An intervertebral prosthesis for implantation into an intervertebral space between a first and a second vertebra of a patient's spinal column, comprising:
a first end;
a second end;
a first bearing surface between the first and second ends, the first bearing surface being configured to bear weight transferred from the first vertebra of the spinal column;
a second bearing surface opposite the first bearing surface, the second bearing surface being configured to transfer weight to the second vertebra of the spinal column;
a first connecting element configured to cooperate with a surgical instrument operable from the first end of the intervertebral prosthesis; and
a second connecting element configured to cooperate with a surgical instrument operable from the second end of the intervertebral prosthesis.

32. The intervertebral prosthesis of claim 31, wherein the first end is opposite the second end.

33. The intervertebral prosthesis of claim 31, wherein second connecting element is on at least one of the first and second bearing surfaces.

34. The intervertebral prosthesis of claim 31, further comprising a keel extending from at least one of the first and second bearing surfaces

35. The intervertebral prosthesis of claim 31, including first and second articulating surfaces configured to articulate the first bearing surface relative to the second bearing surface.

36. The intervertebral prosthesis of claim 35, wherein the articulating surfaces form a ball and socket joint.

37. The intervertebral prosthesis of claim 31, including an elastomeric body disposed between the first and second bearing surfaces.

38. The intervertebral prosthesis of claim 31, wherein the first end includes a leading end of the intervertebral prosthesis, and the second end includes a trailing end of the intervertebral prosthesis.

39. The intervertebral prosthesis of claim 38, wherein the second connecting element is on the leading end.

40. A surgical instrument for implanting the intervertebral prosthesis of claim 39, comprising a connector configured to engage the second connecting element on the leading end.

41. The surgical instrument of claim 40, wherein the surgical instrument is configured to manipulate the intervertebral prosthesis using a pulling motion.

42. The surgical instrument of claim 40, wherein the surgical instrument is configured to removably engage the second connecting element.

43. A surgical instrument for implanting an intervertebral prosthesis in an intervertebral space between a first and a second vertebra of a patient's spinal column, comprising:
a distal end having a connector configured to engage the intervertebral prosthesis and pull the intervertebral prosthesis at least partially within the intervertebral disc space;
a proximal end configured to be manipulated by a physician, wherein the distal end is responsive to the manipulation to pull the intervertebral prosthesis; and
a body extending between the distal and proximal ends.

44. The surgical instrument of claim 43, comprising a hook disposed at the distal end.

45. The surgical instrument of claim 43, wherein the surgical instrument is configured to removable engage the intervertebral disc.

46. The surgical instrument of claim 43, wherein at least a portion of the body is flexible.

47. The surgical instrument of claim 43, wherein it is configured to be operable through a tube.

48. A kit for an intervertebral prosthesis implantable in an intervertebral space between a first and a second vertebra of a patient's spinal column, comprising:
a prosthetic device for insertion into an intervertebral space, the prosthetic device having a first connecting element at a first end and a second connecting element at a second end; and
a first surgical tool for pulling the prosthetic device from the second end.

49. The kit of claim 48, including a second surgical tool for pushing the prosthetic device from the first end.
